(19) United States
(12) Patent Application Publication
(10) Pub. No.: US 2014/0356863 A1
(43) Pub. Date: Dec. 4, 2014

(54) METHODS FOR DETERMINING THE SUSCEPTIBILITY OF A VIRUS TO AN ATTACHMENT INHIBITOR

(71) Applicant: BRISTOL-MYERS SQUIBB COMPANY, Princeton, NJ (US)

(72) Inventors: Mark Krystal, Westport, CT (US); George Hanna, Princeton, NJ (US)

(21) Appl. No.: 14/365,786

(22) PCT Filed: Nov. 19, 2012
(86) PCT No.: PCT/US2012/065806

§ 371 Date: Jun. 16, 2014

Related U.S. Application Data
(60) Provisional application No. 61/721,187, filed on Nov. 1, 2012.
(60) Provisional application No. 61/562,147, filed on Nov. 21, 2011.

(51) Int. Cl. G01N 33/569

(52) U.S. Cl. CPC G01N 33/56988; G01N 2800/52; G01N 2333/161; G01N 2333/162
USPC 435/5

(57) ABSTRACT

The present invention provides novel compositions and methods that can be used for determining the susceptibility of virus to an attachment inhibitor. The present invention also provides methods for predicting whether an infected individual will respond to treatment with an attachment inhibitor, thereby allowing an effective treatment regimen to be designed without subjecting the patient to unnecessary side effects.

### FIELD OF THE INVENTION

This invention relates to methods for determining the susceptibility of a virus to an anti-viral compound, specifically an attachment inhibitor. These methods are useful for identifying effective drug regimens for the treatment of viral infections, and identifying and determining the biological effectiveness of potential therapeutic compounds.

### BACKGROUND OF THE INVENTION

More than 60 million people have been infected with the human immunodeficiency virus (“HIV”), the causative agent of acquired immune deficiency syndrome (“AIDS”), since the early 1980s (Lucas, Lepr. Rev., 73(1):64-71 (2002)). HIV/AIDS is now the leading cause of death in sub-Saharan Africa, and is the fourth biggest killer worldwide. At the end of 2001, an estimated 40 million people were living with HIV globally (Norris, Radiol. Technol., 73(4):339-363 (2002)).

Modern anti-HIV drugs target different stages of the HIV life cycle as well as various enzymes essential for HIV's replication and/or survival. Drugs that have been approved for the treatment of HIV include nucleoside reverse transcriptase (RT) inhibitors: zidovudine (AZT or RETROVIR®), didanosine (VIDEX®), stavudine (ZERIT®), lamivudine (3TC or EPIVIR®), zalcitabine (ddC or HIVID®), abacavir succinate (ZIAGEN®), tenofovir disoproxil fumarate salt (VIREAD®), and emtricitabine (FTC or EMTRIVA®); non-nucleoside RT inhibitors: nevirapine (VIRAMUNE®), delavirdine (RESCRIPTOR®), efavirenz (SUSTIVA®), etravirine (Intelence), and rilpivirine (Edurant); peptidomimetic protease inhibitors: saquinavir (INVIRASE® or Fortovase), indinavir (CRIXIVAN®), ritonavir (NORVIR®), nelfinavir (VIRACEPT®), amprenavir (Agenerase), fosamprenavir (LEXIVA®), lopinavir, darunavir (or Prezista), atazanavir (or REYATAZ®), and tipranavir (APTIVUS®); integrase inhibitors such as raltegravir (Isentress); and entry inhibitors such as enfuvirtide (T-20 or FUZEON®) and maraviroc (Selzentry). Various single pill combinations are also approved, including COMBIVIR® (contains zidovudine plus lamivudine), TRIZIVIR® (contains abacavir, lamivudine, and zidovudine), Epzicom (contains abacavir and lamivudine), TRUVADA® (contains tenofovir and emtricitabine), Atripla (contains tenofovir, emtricitabine, and efavirenz), Complera (contains tenofovir, emtricitabine, and rilpivirine), and KALETRA® (contains lopinavir and ritonavir),

HIV-1 entry is a multistep process and presents numerous opportunities by which compounds can contain virus spread (Kuritzkes, D. R., Curr. Opin. HIVAIDS, 4:82 (2009); Tilton et al., Antiviral Res., 85:91 (2010)). This process is initiated when the gp120 viral envelope protein binds to a well defined locus on the CD4 protein of lymphocytes, leading to a conformational change in gp120 from a pre-CD4 to a CD4-bound state. This interaction induces additional conformational changes in the viral envelope, resulting in co-receptor binding site access (Liu et al., Nature, 455:109 (2008)). While a number of lymphocyte surface proteins can serve as HIV-1 co-receptors, the most commonly utilized receptors are CCRS and CXCR4 (Bazan et al., J. Virol., 72:4485 (1998); Berger et al., Annu. Rev. Immunol., 17:657 (1999); Feng et al., Science, 272:872 (1996)). Co-receptor binding leads to further conformational changes, exposing gp41 and initiating a cell-virus fusion process that results in delivery of the HIV-1 core to the cell cytoplasm (Doms et al., J. Cell Biol., 151:F9 (2000)). Molecules that target any of these defined steps in HIV entry have been designated as entry inhibitors (Kuritzkes, D. R., Curr. Opin. HIVAIDS, 4:82 (2009)).

Attachment inhibitors are a novel subclass of antiviral compounds and target the initial entry step, gp120 binding to the CD4 receptor (Alexander et al., Antimicrob. Agents Chemother., 53:4726 (2009); Guo, et al., J. Virol., 77:10528 (2003); Ho et al., J. Virol., 80:4017 (2006); Kadow et al., Curr. Opin. Investig. Drugs, 7:721 (2006); Lin et al., Proc. Natl. Acad. Sci., 100:11013 (2003)). Since they act upstream of co-receptor binding, attachment inhibitors are active against CCR5 and/or CXCR4 utilizing virus, as well as strains resistant to nucleoside and non-nucleoside reverse transcriptase, protease, and integrase inhibitors (Lin et al., Proc. Natl. Acad. Sci., 100:11013 (2003)). The mechanism underlying attachment inhibitor anti-entry activity likely involves interaction with gp120 in a pre-CD4 conformation and blockage of conformational changes in gp120 that prevent gp120/CD4 contact and subsequent entry events (Ho et al., J. Virol., 80:4017 (2006).

HIV attachment inhibitors are known to exhibit a broad spectrum of antiviral activity against a panel of clinical isolates with, for example, 50% inhibitory concentrations (IC₅₀) (alternatively referred to as 50% effective concentrations (EC₅₀)) ranging over 5-6 log₁₀, from picomolar to greater than 10 μM (Nowicka-Sans, B. et al, Conference Retroviruses Opportunistic Infections Boston, Mass., 2011). This broad inhibitory range is due to heterogeneity observed in the gp120 protein (Kadow et al., Curr. Opin. Investig. Drugs, 7:721 (2006)). Early clinical studies have shown a correlation with IC₅₀ of the virus and clinical response to attachment inhibitors (Hanna et al., Antimicrobial Agents Chemother., 55:722-728 (2011); Nettles, R. et al., Conference Retroviruses Opportunistic Infections Boston, Mass., 2011).

In order to identify patients that would respond best to treatment with an attachment inhibitor, one could first evaluate the sensitivity of the patient's virus to the active agent. However, the classical tests for determining the susceptibility of HIV to an anti-viral agent are complex, time-consuming, expensive, potentially hazardous and not custom tailored to the treatment of a given patient (Barre-Sinoussi et al., Science, 220:868-871 (1983); Popovic et al., Science, 224:497-500 (1984).

Thus, there is a need in the art for a diagnostic assay that could determine whether a patient would be eligible to be treated with a specific attachment inhibitor in a timely and cost efficient manner. The present invention provides such novel assay.

### BRIEF SUMMARY OF THE INVENTION

In one aspect, the invention provides a method of determining the susceptibility of a virus from an infected cell to treatment or inhibition with an attachment inhibitor, the method comprising: (a) isolating virus from a person infected with the virus; (b) adding an attachment inhibitor to the isolated virus; and, (c) measuring the ability of the attachment inhibitor to inhibit binding of a viral protein to CD4. In one embodiment, the attachment inhibitor is a piperazine derivative. In a preferred embodiment, the piperazine derivative is a substituted azaindoleoxoacetic piperazine derivative.

In one embodiment, the attachment inhibitor comprises a linker. In another embodiment, the linker is a quantifiable probe. In a further embodiment, the quantifiable probe comprises a radioactive or nonradioactive label. In another embodiment, the nonradioactive label is a fluorescent label. In a further embodiment, the attachment inhibitor is labeled with a radioactive or nonradioactive label.

In another embodiment, the virus is free virus. In another embodiment, the virus is human immunodeficiency virus (HIV). In a further embodiment, a virus-infected cell is used in place of free virus. In a further embodiment, the infected cell is a T cell or PBMC. In another embodiment, the infected cell is from a person infected with a virus. In another embodiment, the CD4 is added to the isolated virus or infected cell. In another embodiment, the CD4 is purified protein. In another embodiment, the CD4 is on intact cells. In another embodiment, the CD4 comprises a label. In another embodiment, the label is a radioactive or nonradioactive label. In another embodiment, the CD4 is immobilized on a solid surface. In a preferred embodiment, the solid surface is a bead or plastic dish. In another embodiment, the virus is isolated from the serum by binding to an antibody. In a further embodiment, the antibody binds gp160 or p24. In yet another embodiment, the antibody is immobilized on a solid surface.

In another embodiment, the ability of the attachment inhibitor to inhibit binding to CD4 is determined by measuring the amount of bound or unbound virus in the presence and absence of the attachment inhibitor. In a preferred embodiment, the ability of the attachment inhibitor to inhibit binding is determined by immunoassay,

Western blot, PCR or HPLC.

In another aspect, the invention provides a method of determining the susceptibility of a virus from an infected person to treatment with an attachment inhibitor, the method comprising: acquiring a biological sample from a person infected with the virus; isolating the virus from the biological sample; adding an attachment inhibitor to the biological sample; and quantitating the amount of attachment inhibitor bound to a viral protein or the amount of unbound virus.

In another aspect, the invention provides a method of determining the susceptibility of a virus from an infected person to treatment with an attachment inhibitor, the method comprising: acquiring a biological sample from a person infected with the virus; isolating an infected cell from the biological sample; adding an attachment inhibitor to the biological sample; and quantitating the amount of attachment inhibitor bound to a viral protein. In another embodiment, the amount of unbound virus is quantitated.

In another aspect, the invention provides a method of determining the ability of an attachment inhibitor to block gp160 from binding to CD4 as a means of measuring susceptibility of the viral protein to the attachment inhibitor: the method comprising: isolating virus from a person infected with the virus; adding an attachment inhibitor to the isolated virus; adding CD4 to the virus; and, measuring the ability of the attachment inhibitor to inhibit binding of a viral protein to CD4.

In another aspect, the invention provides a method comprising using CD4 binding to virus as a means of isolating virus from a biological sample and determining the effect of adding an attachment inhibitor on the ability of CD4 to capture virus.

In one embodiment, the virus is free virus. In another embodiment, the viral proteins are derived from cells infected with a virus. In another embodiment, the virus is isolated through binding to an antibody. In another embodiment, the antibody binds gp160 or p24. In another embodiment, the antibody is immobilized on a solid surface. In another embodiment, the CD4 comprises a linker. In a preferred embodiment, the quantitation of CD4 or virus is determined by immunoassay, Western blot, PCR or HPLC.

### BRIEF DESCRIPTION OF THE TABLES

Table 1 shows the susceptibility of different HIV-1 strains to treatment with an attachment inhibitor.

### BRIEF DESCRIPTION OF THE FIGURES

FIG. 1 depicts a potential embodiment of the assay. Soluble CD4 protein is bound to a solid matrix and added to serum obtained from an HIV-1 infected individual. The soluble CD4 binds to gp120 on the virus and the complex is removed via the solid matrix and washed. The amount of HIV-1 protein or genomic RNA bound to the matrix is quantitated and compared to the amount of HIV-1 protein or genomic RNA bound in the presence of an attachment inhibitor. The ratio of the two conditions reflects the susceptibility of the gp120 protein for the attachment inhibitor.

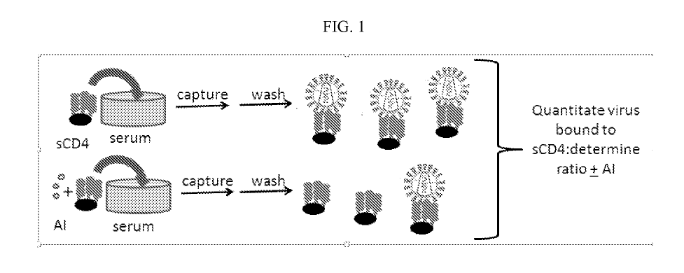

FIG. 2 shows the results of an immunoassay as described in Example 1. Two laboratory strains of HIV-1 were used in this assay, NL4-3, which is susceptible to the attachment inhibitor, the other is RF, which is insensitive to the action of the attachment inhibitor. The amount of virus present with and without attachment inhibitor (AI) is quantitated by a p24 ELISA.

### DETAILED DESCRIPTION OF THE INVENTION

The present invention may be understood more readily by reference to the following detailed description of the preferred embodiments of the invention and the Examples included herein.

The present invention describes methods for determining the susceptibility of a virus to an anti-viral compound, specifically an attachment inhibitor. These methods are useful for identifying effective drug regimens for the treatment of viral infections, and identifying and determining the biological effectiveness of potential therapeutic compounds.

### Definitions

As used herein, “susceptibility” refers to a virus' response to a particular drug. A virus that has decreased or reduced susceptibility to a drug has an increased resistance or decreased sensitivity to the drug, and higher concentrations of the drug are required to demonstrate an effect on the virus. A virus that has increased or enhanced or greater susceptibility to a drug has an increased sensitivity or decreased resistance to the drug, and lower concentrations of the drug are required to demonstrate an effect on the virus.

As used herein, “ICₙ” refers to Inhibitory Concentration. It is the concentration of drug in the patient's blood or in vitro needed to suppress the reproduction of a disease-causing virus (such as HIV) by n %. Thus, “IC₅₀” refers to the concentration of an anti-viral agent at which virus replication is inhibited by 50% of the level observed in the absence of the drug.

As used herein, the term “replication” refers to the process by which a complementary strand of a nucleic acid molecule is synthesized by a polymerase enzyme. In the particular context of the present invention, the term replication as used herein in reference to a virus, refers to the completion of a complete or entire viral life cycle, wherein infectious viral particles or virions attach to the surface of the host cell (usually binding to a specific cell surface molecule that accounts for the specificity of the infection). Once inside the cell, the virions are uncoated and viral genes begin to express leading to the synthesis of proteins needed for replication of the genome and synthesis of new proteins to make new capsids and cores leading to the assembly of progeny infectious virus particles which, themselves, are capable of infecting and replicating in new host cells. Thus, a viral life cycle is only complete if, within a single cell, infection by one or more virus particles or virions proceeds all the way to the production of fully infectious progeny virus particles.

In the particular case of retroviruses, a complete viral life cycle involves infectious viral particles containing the viral RNA entering a cell, the RNA being reverse transcribed into DNA, the DNA being integrated into the host chromosome as a provirus, and the infected cell producing virion proteins and assembling them with full length viral genomic RNA into new, equally infectious particles. “Free virus” refers to virus that is no longer contained within an infected cell but rather is present as a viral particle.

As used herein, the term “viral population” refers to any sample comprising at least one virus, or a collection of virus particles. A sample may be obtained for example from an individual, plant or animal, from cell cultures, or generated using recombinant technology, or cloning. The virus particles of the viral population may all be of the same species and strain, or they may be a mixed population.

As used herein, the term “attachment inhibitor” refers to a molecule that inhibits a virus from the initial attachment of the virus to the surface of the host cell, thereby preventing infection of a cell.

As used herein, the term “HIV attachment inhibitor” refers to a molecule that inhibits HIV from the initial attachment of the virus to the surface of the host cell, thereby preventing infection of a cell. The initial phase of the HIV lifecycle is entry into susceptible host cells. First, the virus attaches to the CD4 cell surface receptor, then it binds to a co-receptor (either CCR5 or CXCR4), and finally it fuses with the cell membrane and merges into the cell. An “HIV attachment inhibitor” may inhibit HIV entry into a cell at the first of these stages where the virus attaches to the CD4 cell surface.

As used herein, the term “piperazine” refers to a broad class of chemical compounds which contain a core piperazine functional group.

As used herein, the term “CD4” refers to “cluster of differentiation 4” which is a glycoprotein expressed on the surface of T helper cells, monocytes, macrophages, and dendritic cells.

As used herein a “non-CD4 protein sequence” or “non-CD4 molecule” is defined as any molecule that does not bind gp120 and does not interfere with the binding of CD4 to its target. An example includes, but is not limited to, an immunoglobulin (Ig) constant region or portion thereof Preferably, the Ig constant region is a human or monkey Ig constant region, e.g., human C(gamma)1, including the hinge, CH2 and CH3 regions. The Ig constant region can be mutated to reduce its effector functions (U.S. Pat. Nos. 5,637,481; and 6,132,992).

As used herein, the term “PBMC” or “PBMCs” are peripheral blood mononuclear cells which refer to any blood cell having a round nucleus. Examples of PBMCs are lymphocytes, monocytes or a macrophages.

As used herein, the term “gp160” refers to an HIV viral protein that forms the viral envelope. During HIV reproduction, the host cell enzymes cleave gp160 into gp120 and gp41 and then gp120 binds to the CD4 receptor on target cells, for example the helper T-cell. The glycoprotein gp41 is non-covalently bound to gp120. When gp120 binds to a CD4 receptor, gp120 undergoes a conformational change thereby exposing gp41 so that it can assist in viral fusion with the host cell.

As used herein, the term “p24” refers to a viral protein that is a component of the HIV particle capsid.

As used herein, the term “antibody” is used in the broadest sense, and specifically covers monoclonal antibodies (including full length monoclonal antibodies), polyclonal antibodies, multispecific antibodies (i.e., bispecific antibodies), and antibody fragments (i.e., Fab, F(ab').sub.2 and Fv) so long as they exhibit binding activity or affinity for a selected antigen. “Antibody” can also refer to an antibody or antibody fragments hanging or fused to carrier proteins/organisms such as phage or other display carriers that have the same properties as isolated antibodies.

As used herein, the terms “peptides”, “proteins” and “polypeptides” are used interchangeably. The “peptides”, “proteins” and “polypeptides” can be generated recombinantly or purified from a cell or serum. The techniques for modifying nucleic acid sequences utilizing recombinant DNA methods to generate recombinant proteins are well known in the art. The term “purified protein” refers to a preparation of a protein or proteins that are preferably isolated from, or otherwise substantially free of, other proteins normally associated with the protein(s) in a cell or cell lysate.

In one aspect, the invention provides a method of determining the susceptibility of virus to treatment or inhibition with an attachment inhibitor, the method comprising: (a) isolating virus or infected cells from a person infected with the virus; (b) adding an attachment inhibitor to the isolated virus or infected cells; and, (c) measuring the ability of the attachment inhibitor to inhibit binding of a viral protein to CD4.

In one embodiment, the virus is HIV. In another embodiment, the attachment inhibitor is an HIV attachment inhibitor compound that is 1-(4-benzoyl-piperazin-1-yl)-2-[4-methoxy-7-(3-methyl-[1,2,4] triazol-1-yl)-1H-pyrralo[2,3-c]pyridine-3-yl]-ethane-1,2-dione, and is set forth and described in U.S. 7,354,924, which is incorporated herein in its entirety:

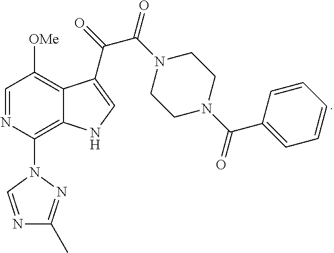 [SMILES: COc1cnc(-n2cnc(C)n2)c2[nH]cc(C(=O)C(=O)N3CCN(C(=O)c4ccccc4)CC3)c12]

The above compound is the parent compound of the prodrug known as 1-benzoyl-4-[2-[4-methoxy-7-(3-methyl-1H-1,2,4-triazol-1-yl)-1-[(phosphonooxy)methyl]-1H-pyrrolo[2,3-c]pyridin-3-yl]-1,2-dioxoethyl]-piperazine.

It is set forth and described in U.S. Pat. No. 7,745,625, which is incorporated by reference herein it its entirety. The compound is represented by the formula below:

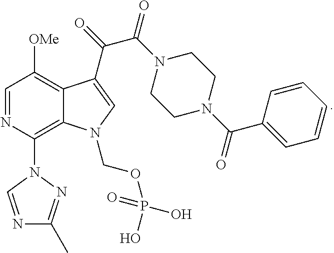 [SMILES: COc1cnc(-n2cnc(C)n2)c2c1c(C(=O)C(=O)N1CCN(C(=O)c3ccccc3)CC1)cn2COP(=O)(O)O]

Various methods for making this prodrug compound are known including those detailed in the '625 reference. In particular, the '625 reference includes various methods for acylation, alkylation and phosphorylation. Another patent reference, U.S. Ser. No. 61/437,821, entitled “METHODS OF MAKING HIV ATTACHMENT INHIBITOR PRODRUG COMPOUND AND INTERMEDIATES”, also details various procedures for making the piperazine prodrug compound. These include a

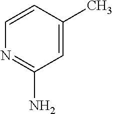

multi-step process which uses the compound as a starting material, which is subsequently brominated, and then nitrated. Further on, a triazolyl moiety is added to the compound before further attaching the piperazine moiety separated by dual carbonyl groups.

In one embodiment, the virus is isolated from plasma, serum, cerebral spinal fluid, breast milk, culture supernatant. In another embodiment, the virus is isolated from serum after release from an infected cell. In another embodiment, the viral proteins are expressed on or within an infected cell. HIV infects vital cells in the human immune system such as helper T cells (specifically CD4⁺ T cells), macrophages, and dendritic cells. Thus, any of these cell types that have been infected with the virus can be utilized. In a preferred embodiment, the infected cell is a T cell or PBMC.

In one embodiment, the virus is isolated from an infected individual through binding to an antibody. Antibodies can bind any viral proteins including but not limited to gp120 and p24. In one embodiment, the antibody is a monoclonal antibody. In another embodiment, the antibody is a polyclonal antibody. In a preferred embodiment, the antibodies are able to react with proteins from all types and subtypes of virus. Antibodies can be produced by methods well known in the art, i.e., by immunizing animals with the viral proteins as antigens. In another embodiment, the antibody is immobilized on a solid surface.

In another aspect, the invention also relates to methods of determining the susceptibility of the virus in an infected cell to treatment with an attachment inhibitor whereby binding of the viral proteins expressed by the infected cell to CD4 is determined by measuring the amount of CD4 bound to the viral protein. Measurement of the susceptibility of the virus to the attachment inhibitor is determined by the ability of the attachment inhibitor to block the binding of CD4 to the viral protein.

In one embodiment, the CD4 is purified protein. In a further embodiment, the CD4 is located on intact cells. In another embodiment, the CD4 is a soluble fusion protein. For example, a soluble CD4 fusion protein such as sCD4-Fc can be produced and isolated via cell cultures.

The cells suitable for culturing a CD4 fusion protein can contain introduced, (e.g., via transformation, transfection, infection, or injection), expression vectors (constructs), such as plasmids and the like, that harbor coding sequences, or portions thereof, encoding the protein of interest for expression and production in the culturing process. Such expression vectors contain the necessary elements for the transcription and translation of the inserted coding sequence. Methods which are well known to and practiced by those skilled in the art can be used to construct expression vectors containing sequences encoding the produced proteins and polypeptides, as well as the appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Control elements, or regulatory sequences, are those non-translated regions of the vector, e.g., enhancers, promoters, 5′ and 3′ untranslated regions, that interact with host cellular proteins to carry out transcription and translation. Such elements can vary in their strength and specificity. Depending on the vector system and host cell utilized, any number of suitable transcription and translation elements, including constitutive and inducible promoters, can be used. In mammalian cell systems, promoters from mammalian genes or from mammalian viruses are preferred. The constructs for use in protein expression systems are designed to contain at least one promoter, an enhancer sequence (optional, for mammalian expression systems), and other sequences as necessary or required for proper transcription and regulation of gene expression (e.g., transcriptional initiation and termination sequences, origin of replication sites, polyadenylation sequences, e.g., the Bovine Growth Hormone (BGH) poly A sequence).

As will be appreciated by those skilled in the art, the selection of the appropriate vector, e.g., plasmid, components for proper transcription, expression, and isolation of proteins produced in eukaryotic (e.g., mammalian) expression systems is known and routinely determined and practiced by those having skill in the art. The expression of proteins by the cells cultured in accordance with the methods of this invention can placed under the control of promoters such as viral promoters, e.g., cytomegalovirus (CMV), Rous sarcoma virus (RSV), phosphoglycerol kinase (PGK), thymidine kinase (TK), or the a-actin promoter. Further, regulated promoters confer inducibility by particular compounds or molecules, e.g., the glucocorticoid response element (GRE) of mouse mammary tumor virus (MMTV) is induced by glucocorticoids (V. Chandler et al., 1983, Cell, 33:489-499). Also, tissue-specific promoters or regulatory elements can be used (G. Swift et al., 1984, Cell, 38:639-646), if necessary or desired.

Expression constructs can be introduced into cells by a variety of gene transfer methods known to those skilled in the art, for example, conventional gene transfection methods, such as calcium phosphate co-precipitation, liposomal transfection, microinjection, electroporation, and infection or viral transduction. The choice of the method is within the competence of the skilled practitioner in the art. It will be apparent to those skilled in the art that one or more constructs carrying DNA sequences for expression in cells can be transfected into the cells such that expression products are subsequently produced in and/or obtained from the cells.

In a particular aspect, mammalian expression systems containing appropriate control and regulatory sequences are preferred for use in protein expressing mammalian cells of the present invention. Commonly used eukaryotic control sequences for use in mammalian expression vectors include promoters and control sequences compatible with mammalian cells such as, for example, the cytomegalovirus (CMV) promoter (CDM8 vector) and avian sarcoma virus (ASV), (πLN). Other commonly used promoters include the early and late promoters from Simian Virus 40 (SV40) (Fiers et al., 1973, Nature, 273:113), or other viral promoters such as those derived from polyoma, Adenovirus 2, and bovine papilloma virus. An inducible promoter, such as hMTII (Karin et al., 1982, Nature, 299:797-802) can also be used.

Examples of expression vectors suitable for eukaryotic host cells include, but are not limited to, vectors for mammalian host cells (e.g., BPV-1, pHyg, pRSV, pSV2, pTK2 (Maniatis); pIRES (Clontech); pRc/CMV2, pRc/RSV, pSFV1 (Life Technologies); pVPakc Vectors, pCMV vectors, pSGS vectors (Stratagene), retroviral vectors (e.g., pFB vectors (Stratagene)), pcDNA-3 (Invitrogen), adenoviral vectors; Adeno-associated virus vectors, baculovirus vectors, yeast vectors (e.g., pESC vectors (Stratagene)), or modified forms of any of the foregoing. Vectors can also contain enhancer sequences upstream or downstream of promoter region sequences for optimizing gene expression.

A selectable marker can also be used in a recombinant vector (e.g., a plasmid) to confer resistance to the cells harboring (preferably, having stably integrated) the vector to allow their selection in appropriate selection medium. A number of selection systems can be used, including but not limited to, the Herpes Simplex Virus thymidine kinase (HSV TK), (Wigler et al., 1977, Cell, 11:223), hypoxanthine-guanine phosphoribosyltransferase (HGPRT), (Szybalska and Szybalski, 1992, Proc. Natl. Acad. Sci. USA, 48:202), and adenine phosphoribosyltransferase (Lowy et al., 1980, Cell, 22:817) genes, which can be employed in tk-, hgprt-, or aprt- cells (APRT), respectively.

Anti-metabolite resistance can also be used as the basis of selection for the following nonlimiting examples of marker genes: dhfr, which confers resistance to methotrexate (Wigler et al., 1980, Proc. Natl. Acad. Sci. USA, 77:357; and O′Hare et al., 1981, Proc. Natl. Acad. Sci. USA, 78:1527); gpt, which confers resistance to mycophenolic acid (Mulligan and Berg, 1981, Proc. Natl. Acad. Sci. USA, 78:2072); neo, which confers resistance to the aminoglycoside G418 (Clinical Pharmacy, 12:488-505; Wu and Wu, 1991, Biotherapy, 3:87-95; Tolstoshev, 1993, Ann. Rev. Pharmacol. Toxicol., 32:573-596; Mulligan, 1993, Science, 260:926-932; Anderson, 1993, Ann. Rev. Biochem., 62:191-21; May, 1993, TIB TECH, 11(5):155-215; and hygro, which confers resistance to hygromycin (Santerre et al., 1984, Gene, 30:147). Methods commonly known in the art of recombinant DNA technology can be routinely applied to select the desired recombinant cell clones, and such methods are described, for example, in Ausubel et al. (eds.), Current Protocols in Molecular Biology, John Wiley & Sons, NY (1993); Kriegler, 1990, Gene Transfer and Expression, A Laboratory Manual, Stockton Press, NY; in Chapters 12 and 13, Dracopoli et al. (eds), Current Protocols in Human Genetics, John Wiley & Sons, NY (1994); Colberre-Garapin et al., 1981. J. Mol. Biol., 150:1, which are incorporated by reference herein in their entireties.

In addition, the expression levels of an expressed protein molecule can be increased by vector amplification (for a review, see Bebbington and Hentschel, “The use of vectors based on gene amplification for the expression of cloned genes in mammalian cells in DNA cloning”, Vol. 3, Academic Press, New York, 1987). When a marker in the vector system expressing a protein is amplifiable, an increase in the level of inhibitor present in the host cell culture will increase the number of copies of the marker gene. Since the amplified region is associated with the protein-encoding gene, production of the protein will concomitantly increase (Crouse et al., 1983, Mol. Cell. Biol., 3:257).

Vectors which harbor glutamine synthase (GS) or dihydrofolate reductase (DHFR) encoding nucleic acid as the selectable markers can be amplified in the presence of the drugs methionine sulphoximine or methotrexate, respectively. An advantage of glutamine synthase based vectors is the availability of cell lines (e.g., the murine myeloma cell line, NSO) which are glutamine synthase negative. Glutamine synthase expression systems can also function in glutamine synthase expressing cells (e.g., CHO cells) by providing additional inhibitor to prevent the functioning of the endogenous gene.

Vectors that express DHFR as the selectable marker include, but are not limited to, the pSV2-dhfr plasmid (Subramani et al., Mol. Cell. Biol. 1:854 (1981). Vectors that express glutamine synthase as the selectable marker include, but are not limited to, the pEE6 expression vector described in Stephens and Cockett, 1989, Nucl. Acids. Res., 17 :7110. A glutamine synthase expression system and components thereof are detailed in PCT publications: WO87/04462; WO86/05807; WO89/01036; WO89/10404; and WO91/06657 which are incorporated by reference herein in their entireties. In addition, glutamine synthase expression vectors that can be used in accordance with the present invention are commercially available from suppliers, including, for example, Lonza Biologics, Inc. (Portsmouth, N.H.).

The CD4 fusion protein of the invention binds the gp120 on target cells, for example the helper T-cell. The CD4 construct has a first amino acid sequence corresponding to the extracellular domain of the CD4 receptor fused to a second amino acid sequence corresponding to the human Ig C.gamma 1 domain. The first amino acid sequence contains one or more of the four immunoglobulin (Ig) like domains (D1-D4) corresponding to the extracellular domain of CD4 joined to a second amino acid sequence containing amino acid residues corresponding to the hinge, CH2 and CH3 regions of human IgC.gamma. 1. In another embodiment, the soluble CD4 molecule includes a junction amino acid residue, which is located between the CD4 portion and the immunoglobulin portion. The junction amino acid can be any amino acid, including glutamine. The junction amino acid can be introduced by molecular or chemical synthesis methods known in the art.

In a particular embodiment, a nucleic acid sequence encoding a soluble CD4 molecule can be inserted into a vector designed for expressing foreign sequences in a eukaryotic host. In this protocol, the DNA fragment encoding the target protein is amplified by PCR, purified, and inserted into an expression vector so as to create a chimeric gene. The vector is then transformed into Escherichia coli and the chimeric DNA isolated and restriction-mapped to test for the presence of the desired insert. DNA from colonies containing the correct insert can then be used to transfect mammalian cells. For example, a fusion gene can be designed in a derivative of the pCDM8 expression vector (Invitrogen #V308-20; UNIT 10.18) that has been modified to contain sequences encoding immunoglobulin constant region IgGl(Fc, hinge, CH2, and CH3 domains of antibody heavy chain) ((Zettlmeissl et al., 1990. Expression and characterization of human CD4:immunoglobulin fusion proteins. DNA Cell Biol.:347-353.). The insert is a fragment of the sequence encoding the extracellular domain of CD4 with incorporated restriction sites XhoI and BamHI.

In another embodiment, the CD4 comprises a label. In another embodiment, the label is a radioactive or nonradioactive label. In another embodiment, the CD4 is immobilized on a solid surface. In a preferred embodiment, the solid surface is a bead or plastic dish.

In another aspect, the ability of the attachment inhibitor to inhibit binding of the virus to CD4 is determined by immunoassay, Western blot, PCR or HPLC. In one embodiment, the immunoassay is a traditional immunoassay including, for example, sandwich immunoassays including ELISA or fluorescence-based immunoassays, as well as other enzyme immunoassays.

In another embodiment, the virus is captured by CD4 that is bound to a solid surface. A surface could be any solid phase surface to which an antibody or protein can be immobilized by covalent linkage, passive absorbance, biotin-strepavidin or any other linkage known to one of ordinary skill in the art. For example, the surface may be a bead, plate, slides, fiber, surface plasmon resonance sensors or any solid surface. In another embodiment, the method is performed in a multi-well plate, nitrocellulose filter or on a glass slide. In one embodiment, a CD4 fusion protein is immobilized on a solid surface. In another embodiment, the CD4 is bound by a CD4 antibody that is immobilized on a solid surface.

In another embodiment, the attachment inhibitor is directly labeled. In a further embodiment, the attachment inhibitor is attached to a linker. The label or linker could be radioactive or nonradioactive. In one embodiment, the label is detected by fluorescence. For example, the label may be selected from the group consisting of phycoerythrin, alexa 532, streptavidin-phycoerythrin and streptavidin-Alexa 532. In another embodiment, the signal is detected by enzymatic activity (i.e., horseradish peroxidase or alkaline phosphatase), chemiluminescence, radioactivity, infrared emission, fluorescence resonance energy transfer (FRET) or any other method known to one of ordinary skill in the art.

In another embodiment, the amount of bound or unbound virus is quantitated. In one embodiment, quantitation is determined by immunoassay, Western blot, PCR or HPLC. In another embodiment, the immunoassay is a traditional immunoassay including, for example, sandwich immunoassays including ELISA or fluorescence-based immunoassays, as well as other enzyme immunoassays.

In another aspect, the invention provides a method of determining the susceptibility of a virus from an infected person to treatment with an attachment inhibitor, the method comprising: acquiring a biological sample from a person infected with the virus; isolating the virus from the biological sample; adding an attachment inhibitor to the biological sample; and quantitating the amount of attachment inhibitor bound to a viral protein or the amount of unbound virus.

In another aspect, the invention provides a method of determining the susceptibility of a virus from an infected person to treatment with an attachment inhibitor, the method comprising: acquiring a biological sample from a person infected with the virus; isolating an infected cell from the biological sample; adding an attachment inhibitor to the biological sample; and quantitating the amount of attachment inhibitor bound to a viral protein or the amount of unbound virus.

In one embodiment, the biological sample is selected from the group consisting of blood, serum, plasma, urine, semen and cerebrospinal fluid. In another embodiment, the amount of virus is quantitated before the attachment inhibitor is added.

In another aspect, the invention provides a method of determining the susceptibility of a virus from an infected cell to treatment or inhibition with an attachment inhibitor, the method comprising: isolating virus from a person infected with the virus; adding an attachment inhibitor to the isolated virus; adding CD4 to the virus; and, measuring the ability of the attachment inhibitor to inhibit binding of a viral protein to CD4.

In one embodiment, the virus is free virus or from a cell infected with a virus. In another embodiment, the virus is isolated through binding to an antibody. In another embodiment, the antibody binds gp160 or p24. In another embodiment, the antibody is immobilized on a solid surface. In a further embodiment, the CD4 comprises a linker.

In another aspect, the invention comprises a kit for conducting an immunoassay to determine the susceptibility of an infected cell to an attachment inhibitor.

### EXAMPLES

### Example 1

### Immunoassay

Two laboratory strains of HIV-1 were used in this assay. One is NL4-3, which is susceptible to the attachment inhibitor and exhibits an IC₅₀ of 2.2±0.6 nM. The other virus strain is the RF strain, which is insensitive to the action of the attachment inhibitor and exhibits an IC₅₀>2000 nM.

Table 1 shows the susceptibility of different HIV-1 strains to treatment with an attachment inhibitor.

| Virus | IC50 (nM) |
|---|---|
| NL4-3 | 2.2 ± 0.6 |
| RF | >2000 |

Virus stock was diluted to 1×10⁴ TCID₅₀ per ml in RPMI media with 10% fetal bovine serum (FBS). The attachment inhibitor (or DMSO) was added to a final concentration of 0.5 uM and the mixture incubated for 15 min at room temperature. Eighty microliters of sCD4-Fc (a recombinant protein containing the gp120 binding domain of CD4 connected to an immunoglobulin Fc receptor) at 5Oug/ml was added and incubated for an additional 30 min, with gentle rocking. Twenty microliters of protein G SEPHAROSE® beads (binding capacity of 200 ug IgG) was added and rocked at 4° C. for an additional 60 min. Unbound virus was removed by washing the beads 4 times with excess PBS. Virus captured on the beads was lysed with 0.5% Triton X-100 and the beads removed by centrifugation. The amount of virus present in the supernatant is quantitated by a p24 ELISA, according to the manufacturer's instructions (PerkinElmer NEK-050).

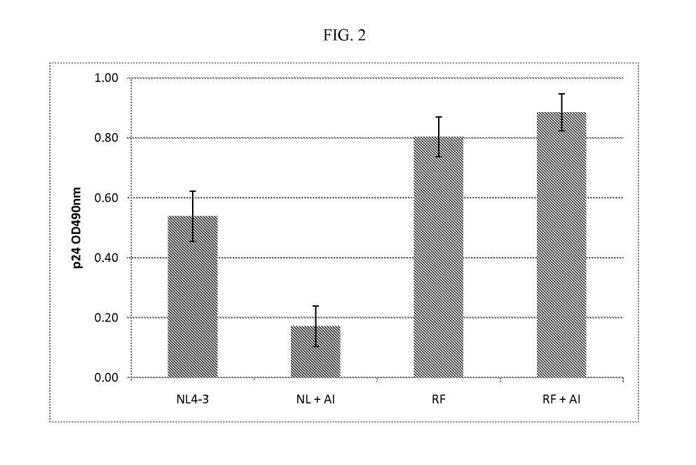

As shown in FIG. 2, the amount of NL4-3 detected when the attachment inhibitor is added is much less than when DMSO is used. However, when the same experiment is performed with the attachment inhibitor-insensitive RF strain, there is no significant difference in the amount of virus captured by the sCD4-Fc construct.

### Example 2

### HIV Isolation from Peripheral Blood Mononuclear Cells

The most preferred source of live HIV is from human peripheral blood samples taken from HIV-infected persons. While HIV may be isolated from a blood sample by any number of procedures, one method of achieving isolation of virus is outlined as follows.

Isolation of HIV is accomplished by first identifying a person infected with HIV and obtaining a whole hepararinized blood sample from them. The blood sample is then centrifuged through Ficol to separate the peripheral blood mononuclear cells (PBM's). The band of PBM's is then removed and the cells washed one time, counted, and resuspended to 1×10⁶ cells/ml. in RPMI 1640 media supplemented with 15% fetal bovine serum and 4 micrograms/ml. PHA. The PBM cells are then cultured in a flask for 48 hours at 37° in a CO₂ -humidified incubator. The cells are then centrifuged and resuspended in media containing 10% IL-2 solution.

Aliquots of fluid plus cells are harvested twice weekly from these cultures and each is tested for the presence of HIV by either reverse transcriptase assay or by HIV antigen-capture assay. These later procedures are well known to those of skill in the art. The cultures may be maintained 4-6 weeks.

### Example 3

### Plasma Virus Isolation

Viral isolates can be obtained from plasma samples using anti-CD44 beads following the manufacturer's protocol (Miltenyi Biotec, Germany). Briefly, before virus extraction, PBMCs from HIV-1-seronegative donors are isolated and CD8⁺ T cells depleted using the RosetteSep human CD8⁺ depletion cocktail (Stemcell Technologies, France). Pooled CD8⁺-depleted PBMCs are then stimulated under three different conditions (‘3×3’ method, Miltenyi Biotech). After 72 hours, cells are mixed to a final concentration of 10⁶ cells/ml in R10 supplemented with IL-2 (100 U/ml) (Roche, Spain), and 200 μl of the extracted virus is added to the culture. Cultures are fed weekly with 10⁶ cells/ml fresh 3×3-stimulated cells. Viral growth is monitored weekly using p24 enzyme-linked immunosorbent assay (ELISA) (Innogenetics, Spain). Virus isolates may be harvested when the p24 concentration in the supernatant reached at least 100 ng/ml and then stored at −80° C.

What is claimed is:

1. A method of determining the susceptibility of a virus from an infected cell to treatment or inhibition with an attachment inhibitor, the method comprising:
(a) isolating virus from a person infected with the virus;
(b) adding an attachment inhibitor to the isolated virus; and
(c) measuring the ability of the attachment inhibitor to inhibit binding of a viral protein to CD4.

2. The method of claim 1, wherein the attachment inhibitor is a piperazine derivative.

3. The method of claim 2, wherein the piperazine derivative is a substituted azaindoleoxoacetic piperazine derivative.

4. The method of claim 1, wherein the attachment inhibitor comprises a linker.

5. The method of claim 4, wherein the linker is a quantifiable probe.

6. The method of claim 5, wherein the quantifiable probe comprises a radioactive or nonradioactive label.

7. The method of claim 6, wherein the nonradioactive label is a fluorescent label.

8. The method of claim 1, wherein the attachment inhibitor is labeled with a radioactive or nonradioactive label.

9. The method of claim 1, wherein the virus is free virus.

10. The method of claim 1, wherein the virus is human immunodeficiency virus (HIV).

11. The method of claim 1, wherein the infected cell is a T cell or PBMC.

12. The method of claim 1, wherein the infected cell is from a person infected with a virus.

13. The method of claim 1, wherein the CD4 is added to the isolated virus.

14. The method of claim 13, wherein the CD4 is purified protein.

15. The method of claim 13, wherein the CD4 is on intact cells.

16. The method of claim 13, wherein the CD4 comprises a label.

17. The method of claim 16, wherein the label is a radioactive or nonradioactive label.

18. The method of claim 13, wherein the CD4 is immobilized on a solid surface.

19. The method of claim 18, wherein the solid surface is a bead or plastic plate.

20. The method of claim 1, wherein the ability of the attachment inhibitor to inhibit binding to CD4 is determined by measuring the amount of bound or unbound virus in the presence and absence of the attachment inhibitor.

21. The method of claim 1, wherein the ability of the attachment inhibitor to inhibit binding is determined by immunoassay, Western blot, PCR or HPLC.

22. A method of determining the susceptibility of a virus from an infected person to treatment with an attachment inhibitor, the method comprising:
(a) acquiring a biological sample from a person infected with the virus;
(b) isolating the virus from the biological sample;
(c) adding an attachment inhibitor to the biological sample; and
(d) quantitating the amount of attachment inhibitor bound to a viral protein or the amount of unbound virus.

23. A method of determining the susceptibility of a virus from an infected person to treatment with an attachment inhibitor, the method comprising:
(a) acquiring a biological sample from a person infected with the virus;
(b) isolating an infected cell from the biological sample;
(c) adding an attachment inhibitor to the biological sample; and
(d) quantitating the amount of attachment inhibitor bound to a viral protein or the amount of unbound virus.

24. The method of claim 22 or 23, wherein the biological sample is selected from the group consisting of blood, serum, plasma, urine, semen and cerebrospinal fluid.

25. The method of claim 22, wherein the amount of virus is quantitated before the attachment inhibitor is added.

26. A method of determining the susceptibility of an infected cell to treatment or inhibition with an attachment inhibitor, the method comprising:
(a) isolating virus from a person infected with the virus;
(b) adding an attachment inhibitor to the isolated virus;
(c) adding CD4 to the virus; and
(d) measuring the ability of the attachment inhibitor to inhibit binding of a viral protein to CD4.

27. The method of claim 26, wherein the virus is free virus or from a cell infected with a virus.

28. The method of claim 26, wherein the virus is isolated through binding to an antibody.

29. The method of claim 28, wherein the antibody binds gp160 or p24.

30. The method of claim 28, wherein the antibody is immobilized on a solid surface.

31. The method of claim 27, wherein the CD4 comprises a linker.
